(19) United States
(12) Patent Application Publication
(10) Pub. No.: US 2024/0229751 A1
(43) Pub. Date: Jul. 11, 2024

(54) INJECTION NEEDLE BLOWOFF APPARATUS AND INJECTION TESTING SYSTEMS

(71) Applicant: Illinois Tool Works Inc., Glenview, IL (US)

(72) Inventor: Ryan Levy, Boston, MA (US)

(21) Appl. No.: 18/150,282

(22) Filed: Jan. 5, 2023

(51) Int. Cl. F02M 47/02; A61M 5/32

(52) U.S. Cl. CPC F02M 47/02; A61M 5/32; A61M 5/46

(57) ABSTRACT

Disclosed example injection needle blowoff apparatus include: a loading surface having a first side configured to contact an injector and having a second side opposite the first side; and an adjustable blowoff nozzle adjacent the second side of the loading surface, the adjustable blowoff nozzle comprising: a gas inlet configured to be coupled to a gas supply; and a gas outlet configured to direct gas from the gas inlet towards a location of a needle of the injector, the gas outlet being adjustable to blow on the needle within a range of distances from the second side of the loading surface.

### FIELD OF THE DISCLOSURE

This disclosure relates generally to injection device testing, and more particularly, to injection needle blowoff apparatus and injection testing systems.

### BACKGROUND

Injection testing systems may test one or more aspects of injection devices, including autoinjectors, for aspects such as cap removal force, plunger actuation force, injection depth, needle retraction, and/or delivered dose.

### SUMMARY

Injection needle blowoff apparatus and injection testing systems are disclosed, substantially as illustrated by and described in connection with at least one of the figures, as set forth more completely in the claims.

### BRIEF DESCRIPTION OF THE DRAWINGS

These and other features, aspects, and advantages of the present disclosure will become better understood when the following detailed description is read with reference to the accompanying drawings in which like characters represent like parts throughout the drawings, wherein:

FIG. 1 is an example injection testing system to perform testing of injection devices, in accordance with aspects of this disclosure.

FIG. 2 is a block diagram of an example injection testing system including an injection needle blowoff apparatus, in accordance with aspects of this disclosure.

FIG. 3 is a perspective view of an example implementation of elements of the injection testing system of FIG. 2.

FIG. 4 is a perspective view of the example injection needle blowoff apparatus of FIG. 3 including a positioning plate and blowoff nozzle.

FIG. 5 is a front elevation view of the example injection needle blowoff apparatus of FIG. 3.

FIG. 6 is a bottom plan view of the example injection needle blowoff apparatus of FIG. 3.

FIGS. 7A and 7B are different perspective views of the example injection needle blowoff apparatus of FIG. 3.

FIG. 8 is a front section view of the example injection needle blowoff apparatus of FIG. 3.

FIG. 9A illustrates another example implementation of the blowoff nozzles, in which the blowoff nozzles each have an adjustable distance from the tip of the needle in a direction parallel to the needle.

FIG. 9B illustrates another example implementation of the blowoff nozzles, in which the blowoff nozzles each have an adjustable angle to adjust an angle of the gas outlet with respect to the bottom surface of the positioning plate.

The figures are not necessarily to scale. Wherever appropriate, similar or identical reference numerals are used to refer to similar or identical components.

### DETAILED DESCRIPTION

Injection needle testing devices that measure the dosage delivered by the injection needle, particularly by autoinjectors, include a flask or other container positioned to capture and measure the fluid expelled from the needle. To accurately measure the expelled fluid, injection needle testing devices may include a blowoff or other method to detach the final quantity of expelled fluid, which can tend to remain adhered to the needle by fluid adhesion. While injection devices may have a wide range of geometries and dimensions, conventional injection needle testing devices have a limited range of testable needle lengths due at least in part to the structure of the blowoff device. Changing the needle length to be tested can involve a relatively lengthy process of changing the blowoff.

Disclosed example injection needle blowoff apparatus and injection testing systems provide a wider range of needle lengths that can be tested using a same blowoff apparatus. In some disclosed examples, the exposed length of needle that can be tested is 2 mm or more, including blowing off the last drop of expelled fluid from the injection needle. In some examples, the position(s) and/or orientation(s) of one or more blowoff nozzles are adjustable with respect to a position of the needle. By adjusting the position(s) and/or orientations of the blowoff nozzle(s), the location at which the gas delivered by the blowoff nozzle can be tailored to the needle length and the resulting location of the last drop of expelled fluid.

Disclosed example injection needle blowoff apparatus include: a loading surface having a first side configured to contact an injector and having a second side opposite the first side; and an adjustable blowoff nozzle adjacent the second side of the loading surface, the adjustable blowoff nozzle including: a gas inlet configured to be coupled to a gas supply; and a gas outlet configured to direct gas from the gas inlet towards a location of a needle of the injector, the gas outlet being adjustable to blow on the needle within a range of distances from the second side of the loading surface.

In some example injection needle blowoff apparatus, the adjustable blowoff nozzle is configured to have an adjustable distance from the aperture along a plane of the second side of the loading surface. In some example injection needle blowoff apparatus, the gas outlet is configured to blow at an angle away from the second side of the loading surface, and the gas outlet is configured to blow on the needle at the location based on the distance between the adjustable blowoff nozzle and the needle in in a plane perpendicular to the needle.

In some example injection needle blowoff apparatus, the adjustable blowoff nozzle is configured to have an adjustable distance from a tip of the needle in a direction parallel to the needle. In some example injection needle blowoff apparatus, the loading surface includes a positioning plate having an aperture, extending from a first side of the positioning plate to a second side of the positioning plate. In some example injection needle blowoff apparatus, the adjustable blowoff nozzle is positioned on a first side of the aperture, and further includes a second adjustable blowoff nozzle positioned on a second side of the aperture. In some example injection needle blowoff apparatus, the second adjustable blowoff nozzle includes: a second gas inlet configured to be coupled to the gas supply; and a second gas outlet configured to direct the gas towards the location of the needle, the second gas outlet being adjustable to blow on the needle within the range of distances. In some example injection needle blowoff apparatus, the aperture has at least one dimension smaller than a corresponding dimension of a body of the injector.

In some example injection needle blowoff apparatus, the adjustable blowoff nozzle is configured to have an adjustable angle of the gas outlet. In some example injection needle blowoff apparatus, the adjustable blowoff nozzle includes a body defining a channel between the gas inlet and the gas outlet, and the channel is configured to increase at least one of a flow speed of the gas or a pressure of the gas between the gas inlet and the gas outlet. Some example injection needle blowoff apparatus further include control circuitry configured to automatically control a blowoff actuator to adjust a location of the blowoff nozzle.

Some disclosed injector testing devices include: a gas supply; an injection needle blowoff device, including: a loading surface having a first side configured to contact an injector and having a second side opposite the first side; an adjustable blowoff nozzle adjacent to the second side of the loading surface, the adjustable blowoff nozzle including: a gas inlet configured to be coupled to the gas supply; and a gas outlet configured to direct gas from the gas inlet towards a location of a needle of the injector, the gas outlet being adjustable to blow on the needle within a range of distances from the second side of the loading surface; and control circuitry configured to control the gas supply to output the gas to the adjustable blowoff nozzle.

Some example injector testing devices further include an injector positioner configured to position the injector, in which the control circuitry is configured to control the injector positioner to position the injector. In some example injector testing devices, the injector positioner is configured to move a body of the injector into contact with the loading surface.

Some example injector testing devices further include an injector actuator configured to actuate the injector to expel contents of the injector via the needle while the needle of the injector is adjacent the adjustable blowoff nozzle, in which the control circuitry is configured to control the injector actuator to actuate the injector. In some example injector testing devices, the injector actuator is configured to actuate the injector when the body of the injector is in contact with the loading surface.

Some example injector testing devices further include a collection container configured to collect the contents expelled from the injector. In some example injector testing devices, the loading surface includes a positioning plate having an aperture, extending from a first side of the positioning plate to a second side of the positioning plate, and wherein the adjustable blowoff nozzle is configured to have an adjustable distance from the aperture along a plane of the second side of the positioning plate, the gas outlet is configured to blow at an angle away from the second side of the positioning plate, and the gas outlet is configured to blow on the needle at the location based on the distance between the adjustable blowoff nozzle and the distance from the aperture in a direction parallel to the second side of the positioning plate.

In some example injector testing devices, the adjustable blowoff nozzle is configured to have an adjustable distance from a tip of the needle in a direction parallel to the needle. Some example injector testing devices further include a second adjustable blowoff nozzle configured to direct gas towards the needle. In some example injector testing devices, the gas supply comprises a compressed gas source, a pneumatic pump, or a blower.

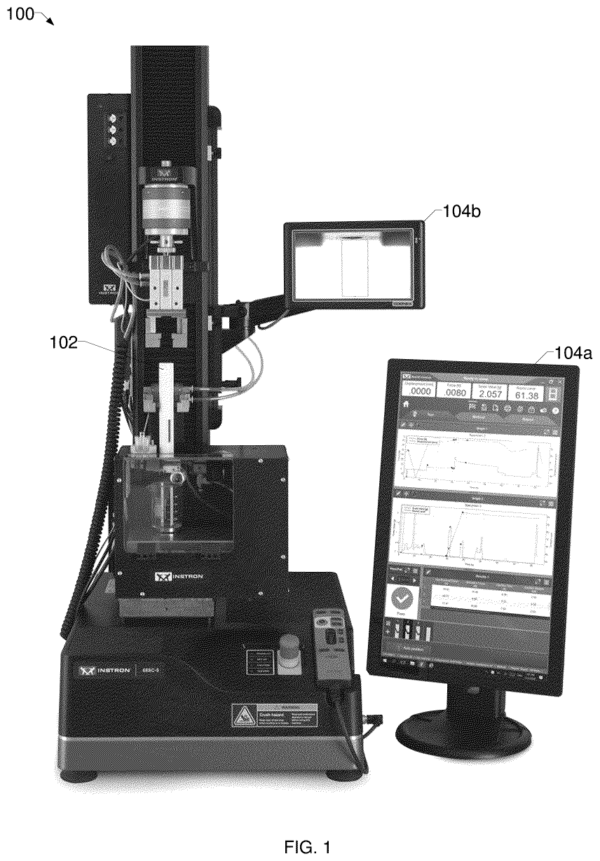

FIG. 1 is an example injection testing system 100 to perform testing on injection devices, such as an autoinjector 102. The example injection testing system 100 may be configured, for example, to perform some or all tests to evaluate requirements of the ISO 11608-5 standard. The example injection testing system 100 may be, for example, a universal testing system configured for injection testing.

The example injection testing system 100 of FIG. 1 includes positioning device(s) (e.g., to position the autoinjector 102 in one or more positions and/or orientations for automated testing), actuator(s) (e.g., to actuate components of the autoinjector 102, to actuate the positioning device(s), to position and/or orient test devices, etc.), and/or sensors to measure aspects of the autoinjector 102 during testing (e.g., load sensors to measure actuation force(s), auditory sensors to detect audible events), mass scales to measure an expelled dose, displacement and/or position sensors to trigger testing steps and/or measure displacement of components of the autoinjector 102, etc.). The example injection testing system 100 further includes one or more user interface devices, such as displays 104a, 104b and input devices 106.

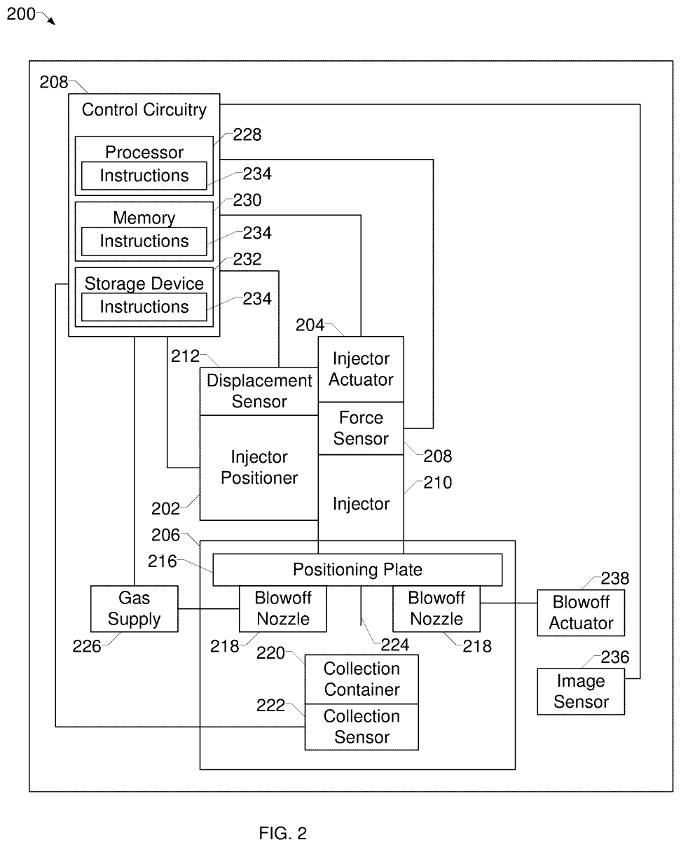

FIG. 2 is a block diagram of an example injection testing system 200 including an injection needle blowoff apparatus. The example injection testing system 200 may be used to implement some or all of the components of the injection testing system 100 of FIG. 1.

The example injection testing system 200 includes an injector positioner 202, an injector actuator 204, an injection collector 206, and control circuitry 208. The injector positioner 202 positions and/or orients an injector 210 (e.g., an autoinjector) for one or more tests in the injection testing system 200. For example, the injector positioner 202 may grasp the injector 210 and move and/or rotate the injector 210 for testing. The injector positioner 202 and/or the position of the injector 210 may be measured by one or more displacement sensor(s) 212, which provides displacement and/or position information to the control circuitry 208.

The injector actuator 204 actuates one or more aspects of the injector 210, such as a plunger or other injection mechanism of the injector 210. The force applied by the injector actuator 204 may by measured by a force sensor 214, which provides force measurements to the control circuitry 208.

The injection collector 206 includes a loading surface (e.g., a positioning plate 216), blowoff nozzles 218, and a collection container 220. The collection container 220 and the injector 210 are positioned such that, when the injector 210 is actuated to expel fluid contained in the injector 210, the fluid is expelled into the collection container 220. A collection sensor 222 measures the mass and/or volume collected in the collection container 220, and provides a mass or volume measurement to the control circuitry 208.

The positioning plate 216 allows a needle 224 of the injector 210 to extend through the positioning plate 216 toward the collection container 220. The positioning plate 216 may block the body 225 of the injector 210 from extending through the positioning plate 216 using appropriately sized apertures for the needle 224 and body 225 of the injector 210. To test the dispensing of the contained fluid, the injector positioner 202 may position the injector 210 to contact or abut the positioning plate 216, such that the needle 224 extends through the aperture of the positioning plate 216. When the injector 210 is positioned, the injector 210 may be actuated (e.g., manually, or automatically via the injector actuator 204) to expel the contents of the injector 210 into the collection container 220.

While the examples disclosed herein use the positioning plate 216 as a loading surface, other examples may use different types of loading surfaces against which the injector 210 can be actuated to expose the needle 224 and/or expel the contents of the injector 210. For example, rods or other structural members which are positioned to contact a body of the injector 210 on a top side of the loading surface, and to avoid obstructing the needle 224 may be used. In some such examples, the blowoff nozzles 218 may be coupled to another surface, or otherwise adjustably supported, within the injection collector 206 adjacent the bottom side of the loading surface and/or adjacent the location the needle 224.

At the completion of actuation of the injector 210, the blowoff nozzles 218 are controlled to blow the last drop of fluid from at or near the tip of the needle into the collection container 220. A gas supply 226 provides a gas, such as nitrogen or air, to the blowoff nozzles 218. The gas supply 226 may be, for example, a compressed gas source, a pneumatic pump, or a blower. As disclosed in more detail below, the blowoff nozzles 218 may be positioned and/or oriented to adjust a location at which the blowoff gas strikes the needle 224.

The example control circuitry 208 may be a general-purpose computer, a laptop computer, a tablet computer, and/or any other type of processing system configured to communicate with the sensors and actuators of the injection testing system 200. For example, the control circuitry 208 includes a processor 228, memory 230, and a storage device 232. The example processor 228 may be any general purpose central processing unit (CPU) from any manufacturer. In some other examples, the processor 228 may include one or more specialized processing units, such as RISC processors with an ARM core, graphic processing units, digital signal processors, and/or system-on-chips (SoC). The processor 228 executes machine readable instructions 234 that may be stored locally at the processor (e.g., in an included cache or SoC), in the memory (e.g., a random access memory or other volatile memory, a read only memory or other non-volatile memory such as FLASH memory, and/or in the storage device 232. The example storage device 232 may be a hard drive, a solid state storage drive, a hybrid drive, a RAID array, and/or any other mass data storage device.

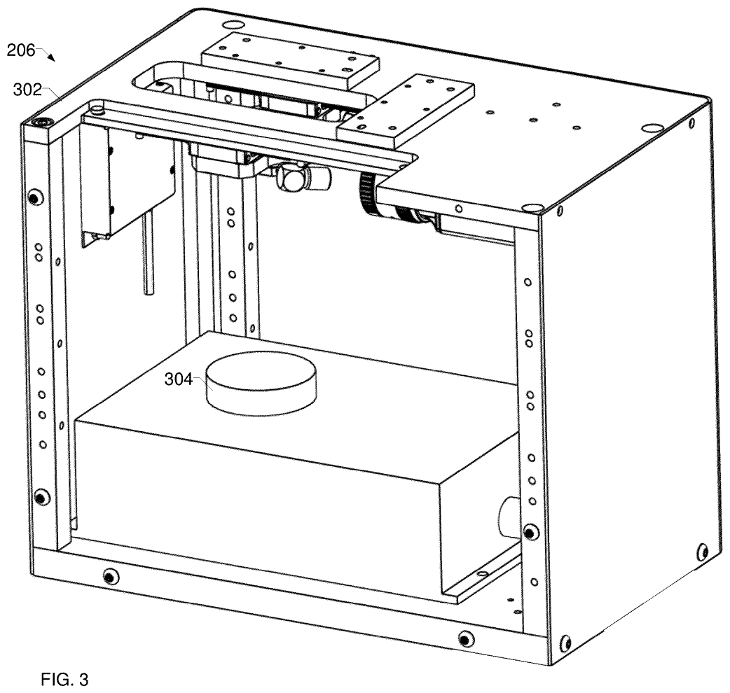

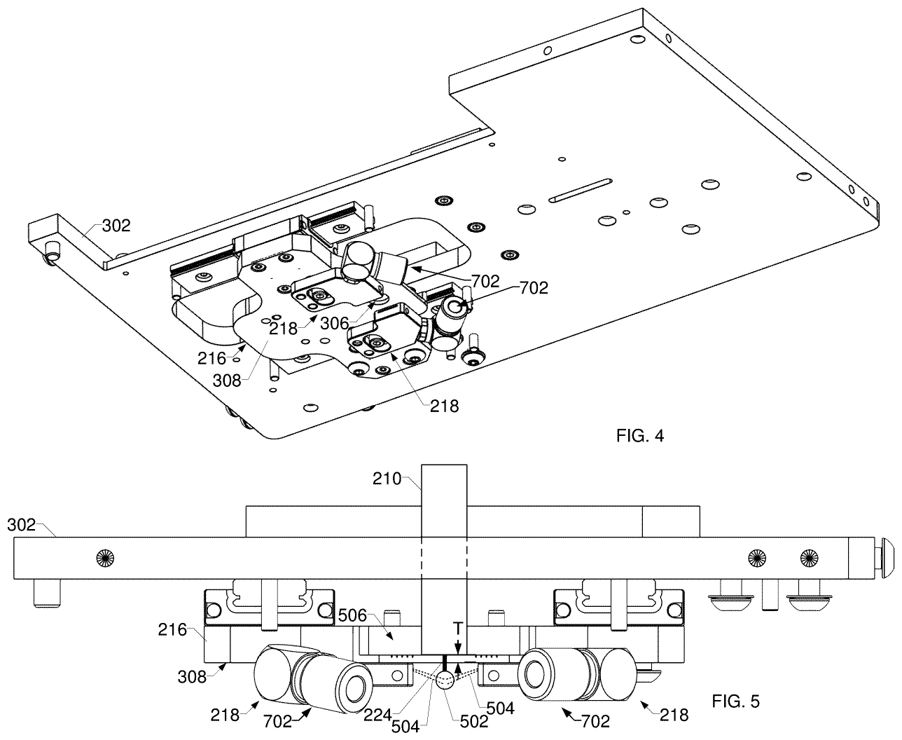

FIG. 3 is a perspective view of an example implementation of the example injection collector 206 of FIG. 2. The injection collector 206 includes a housing 302, within which is located the positioning plate 216, blowoff nozzles 218, and a mass scale 304 (e.g., collection sensor 222 of FIG. 2). FIG. 4 is a perspective view of the example injection needle blowoff apparatus of FIG. 3 including a positioning plate 216 and blowoff nozzle 218. FIG. 5 is a front elevation view of the example injection needle blowoff apparatus of FIG. 2.

The positioning plate 216 is positioned below a top part of the housing 302, such that a top surface of the positioning plate 216 is accessible by the injector 210 through the housing 302. The positioning plate 216 is coupled to the housing 302, and may be replaced with other positioning plates to test different types of injectors (e.g., having different needle lengths, having different body dimensions). While the blowoff nozzles 218 are adjustable as disclosed in more detail below, replacement of the positioning plate 216 and attached blowoff nozzles 218 may allow more rapid changes to accommodate different testing procedures for different injectors.

The blowoff nozzles 218 are coupled to a bottom surface 308 of the positioning plate 216 on opposite sides of an aperture 306 in the positioning plate 216. The needle 224 of the injector 210 extends through the aperture 306 and sticks out the bottom side of the positioning plate 216. When the injector 210 is actuated, fluid is expelled from the needle 224 into the collection container 220 (e.g., positioned on the mass scale 304 below the needle 224).

As illustrated in FIG. 5, at the end of the expulsion of the fluid, the last quantity of the fluid tends to adhere to the needle 224 in the form of a drop 502. After the actuation is completed, the control circuitry 208 controls the gas supply 226 and/or the blowoff nozzles 218 (e.g., via a valve or other control device) to blow gas 504 toward the needle 224 to dislodge the drop 502 into the collection container 220.

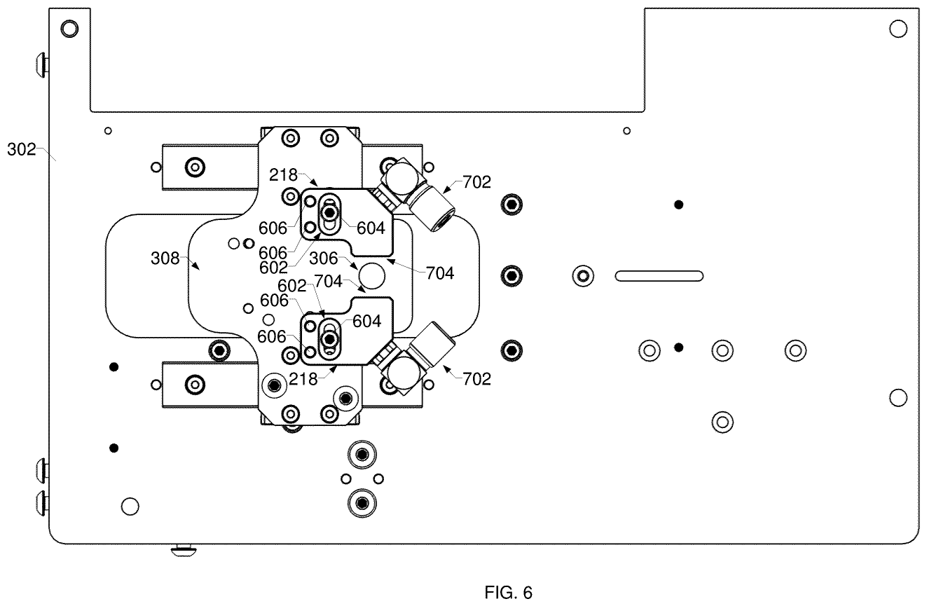

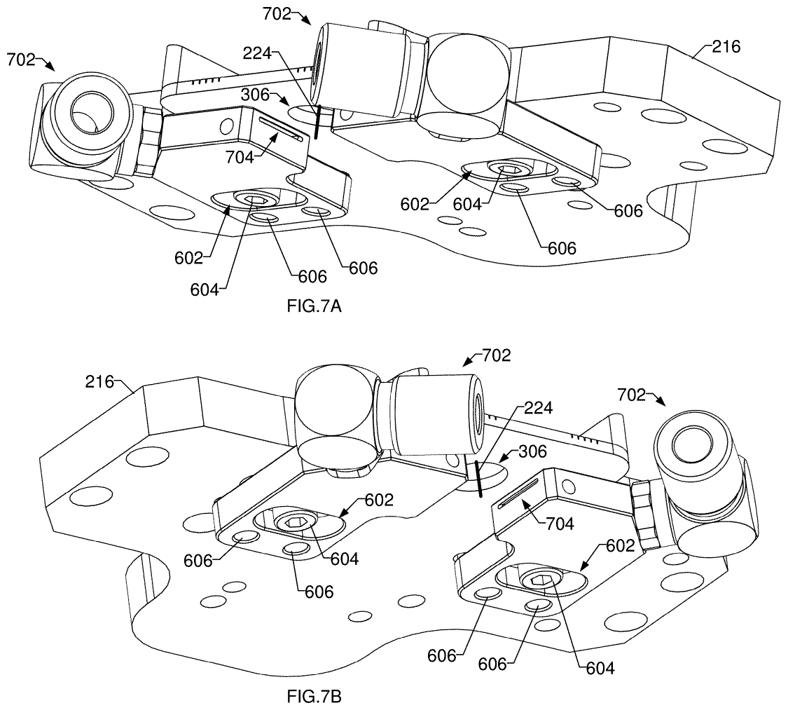

FIG. 6 is a bottom plan view of the example injection needle blowoff apparatus of FIG. 2. FIGS. 7A and 7B are different perspective views of the example injection needle blowoff apparatus of FIG. 3, including the positioning plate 216 and the blowoff nozzles 218.

The example blowoff nozzles 218 each include a gas inlet 702, which are coupled to the gas supply 226 via a hose or other connection. As shown in FIGS. 7A and 7B, the blowoff nozzles 218 also include a gas outlet 704, which directs the gas received via the corresponding gas inlet 702 towards the location of the needle 224.

The example gas outlets 704 of FIGS. 7A and 7B direct the gas at least partially away from the bottom surface of the positioning plate 316. The blowoff nozzles 218 are also adjustable, such that the gas outlets 704 can be adjusted to direct the gas 504 toward the tip of the needle 224 for a range of needle lengths. In the example of FIGS. 4-7B, the blowoff nozzles 218 have an adjustable distance (e.g., in the direction toward and away from the aperture 306 of the positioning plate 216) along a plane of the bottom surface 308 of the positioning plate 216 (e.g., parallel to the bottom surface of the positioning plate 216, within a plane perpendicular to the needle 224). To this end, each of the blowoff nozzles 218 includes a slot 602, a screw 604, and dowels 606, to allow for adjustment of the position of the blowoff nozzles 218 and to secure the blowoff nozzles 218 in a desired position.

The screw 604 extends through the slot 602 and into a threaded hole in the positioning plate 216. The slot 602 extends in a direction of adjustment of the blowoff nozzle 218, such that the blowoff nozzle 218 may slide toward and away from the aperture 306 when the screw 604 is loosened from the positioning plate 216. When the blowoff nozzle 218 is positioned in the desired location, the screw 604 may be tightened to clamp the blowoff nozzle 218 to the positioning plate 216. The example screw 604 may be replaced with a dowel that extends from the positioning plate 216 into the slot 602, and a different clamping mechanism, such as a clamp or clip coupled to the positioning plate 216, to clamp or otherwise secure the blowoff nozzle 218 in a desired position.

The dowels 606 extend from the blowoff nozzles 218 into a corresponding slot in the positioning plate 216. The slot in the positioning plate 216 extends parallel to the slot 602 in the blowoff nozzles 218, and limit rotation of the blowoff nozzles 218 (e.g., to keep the gas outlets 704 directed towards the needle 224). The dowels 606 may be replaced with another rotation limiting device, such as a bracket coupled to the positioning plate 216 adjacent the blowoff nozzles 218 to limit rotation and/or guide movement of the blowoff nozzles 218. In some examples, the blowoff nozzles 218 may be coupled to one or more respective worm gears, rack and pinion gears, and/or other gearing systems, which may then be actuated to adjust the positioning of the blowoff nozzles 218.

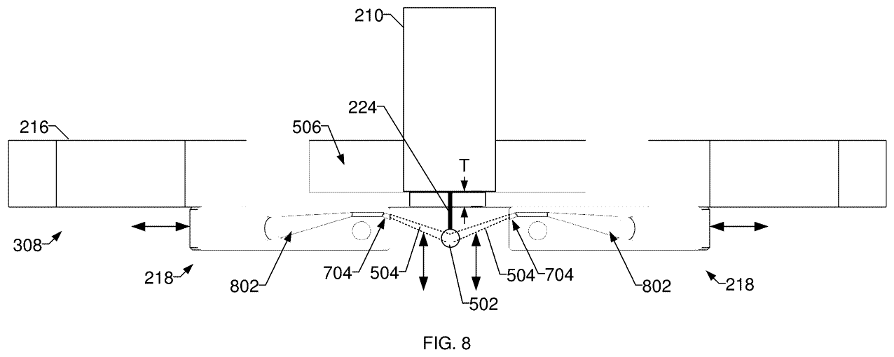

FIG. 8 is a front section view of the example blowoff nozzles 218 of FIGS. 4-7B. As illustrated in FIG. 8, the example blowoff nozzles 218 include passages 802 extending from the gas inlets 702 toward the gas outlets 704. The cross-sectional area of the passages 802 decreases from the gas inlet 702 to the gas outlet 704 to increase the speed of the gas 504.

As shown in FIG. 8, the passage 802 directs the gas 504 away at least partially away from the bottom surface 308 of the positioning plate 216 as the gas 504 exits the gas outlet 704. As the blowoff nozzle 218 and the gas outlet 704 is adjusted (e.g., via the screw 604 and the slot 602) to be closer to the needle 224, the distance between the bottom surface 308 and the location on the needle 224 decreases due to the angle of the passage 802 at the gas outlet 704. Conversely, as the blowoff nozzle 218 and the gas outlet 704 is adjusted to be farther from the needle 224, the distance between the bottom surface 308 and the location on the needle 224 increases.

The example blowoff nozzles 218 may be constructed using additive manufacturing or 3D printing, and/or using conventional reductive manufacturing techniques, to form the passage 802.

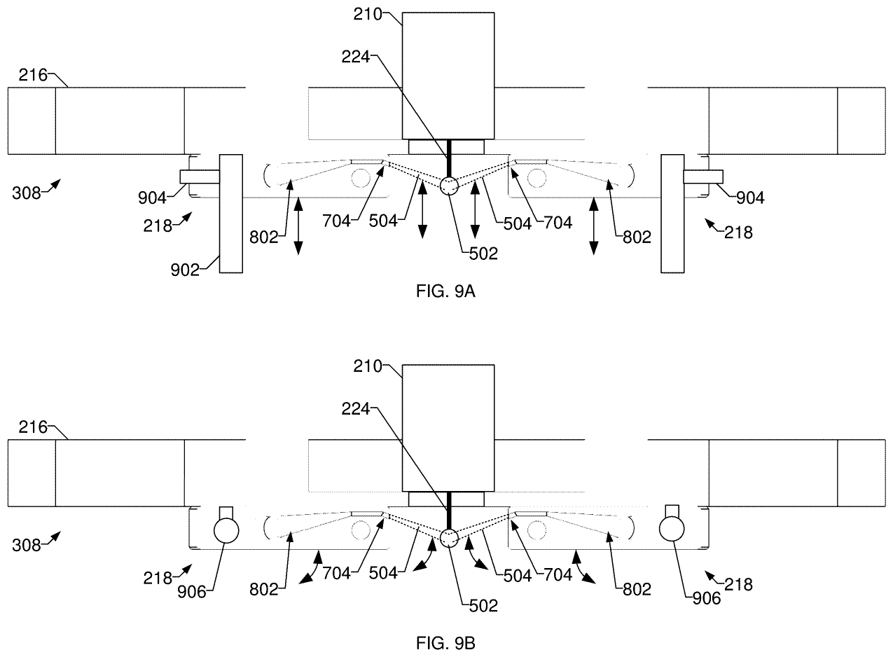

FIG. 9A illustrates another example implementation of the blowoff nozzles 218, in which the blowoff nozzles 218 each have an adjustable distance from the tip of the needle 224 in a direction parallel to the needle 224 (e.g., an adjustable distance from the bottom surface 308 of the positioning plate 216. In the example of FIG. 9A, the blowoff nozzle 218 includes the gas inlet 702 and the gas outlet 704. Instead of, or in addition to, being adjustable in a direction parallel to the bottom surface 308 of the positioning plate 216, the blowoff nozzles of FIG. 9A are adjustable along a dowel 902 that is parallel to the example needle 224. When the blowoff nozzle 218 is adjusted to the desired position, a set screw 904 may secure the blowoff nozzle 218 to the desired distance from the bottom surface 308. The location at which the gas 504 interfaces with the needle 224 is adjusted by adjusting the position of the blowoff nozzle 218.

In some other examples, a helical rod or other support structure may be used to support the blowoff nozzle 218 instead of the dowel. The set screw 904 may likewise be replaced with a pin, a clamp, or other securing device.

FIG. 9B illustrates another example implementation of the blowoff nozzles 218, in which the blowoff nozzles 218 each have an adjustable angle to adjust an angle of the gas outlet 704 with respect to the bottom surface of the positioning plate 216. In the example of FIG. 9B, the blowoff nozzle 218 includes the gas inlet 702 and the gas outlet 704.

The example blowoff nozzles 218 of FIG. 9B are coupled to the positioning plate 216 by a ball joint 906 or other rotating joint such as a hinge. In some examples, the ball joint 906 may be geared or otherwise controlled to set the blowoff nozzles 218 at a desired angle. The angle of the gas outlet 704 and the gas 504 and, as a result, the position on the needle 224 at which the gas 504 interfaces with the needle 224, is adjustable by adjusting the angle of the blowoff nozzles 218.

As illustrated in FIGS. 5 and 8, the thickness T of the positioning plate 216 is decreased in an area 506 around the aperture 306, as compared with the remainder of the positioning plate 216. By reducing the thickness of the area 506 and/or adjusting the position of the blowoff nozzles 218 with respect to the aperture 306, the range of needle lengths that can be tested is increased. For example, the example positioning plate 216 and blowoff nozzles 218 may be used to perform tests on needle lengths (measuring the exposed length of the needle 224) down to 2 mm long.

The example blowoff nozzles 218 disclosed herein may be adjusted manually or automatically. For example, the blowoff nozzles 218 may be coupled to a motor, a gearing system, and/or other actuation device to control the positioning and/or orientation of the blowoff nozzles 218. In some examples, the injection testing system 200 may include a needle detection sensor, such as an image sensor 236, that determines the length of the needle 224 and/or the positioning of the tip of the needle 224. For example, the image sensor 236 may determine the position of the tip of the needle 224 by analyzing an image of the positioned needle 224. Based on the determined tip, the control circuitry 208 automatically controls the position and/or orientation of the blowoff nozzles 218, using a blowoff actuator 238 coupled to the blowoff nozzles 218, to direct the gas 504 toward the location of the tip of the needle 224.

While disclosed examples include two blowoff nozzles on opposing sides of the needle, other examples may include a single blowoff nozzle, or three or more blowoff nozzles.

The present methods and systems may be realized in hardware, software, and/or a combination of hardware and software. The present methods and/or systems may be realized in a centralized fashion in at least one computing system, or in a distributed fashion where different elements are spread across several interconnected computing systems. Any kind of computing system or other apparatus adapted for carrying out the methods described herein is suited. A typical combination of hardware and software may include a general-purpose computing system with a program or other code that, when being loaded and executed, controls the computing system such that it carries out the methods described herein. Another typical implementation may comprise an application specific integrated circuit or chip. Some implementations may comprise a non-transitory machine-readable (e.g., computer readable) medium (e.g., FLASH drive, optical disk, magnetic storage disk, or the like) having stored thereon one or more lines of code executable by a machine, thereby causing the machine to perform processes as described herein. As used herein, the term “non-transitory machine-readable medium” is defined to include all types of machine readable storage media and to exclude propagating signals.

As utilized herein the terms “circuits” and “circuitry” refer to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. In other words, “x and/or y” means “one or both of x and y”. As another example, “x, y, and/or z” means any element of the seven-element set {(x), (y), (z), (x, y), (x, z), (y, z), (x, y, z)}. In other words, “x, y and/or z” means “one or more of x, y and z”. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” to perform a function whenever the circuitry comprises the necessary hardware and code (if any is necessary) to perform the function, regardless of whether performance of the function is disabled or not enabled (e.g., by a user-configurable setting, factory trim, etc.).

While the present method and/or system has been described with reference to certain implementations, it will be understood by those skilled in the art that various changes may be made and equivalents may be substituted without departing from the scope of the present method and/or system. For example, block and/or components of disclosed examples may be combined, divided, re-arranged, and/or otherwise modified. In addition, many modifications may be made to adapt a particular situation or material to the teachings of the present disclosure without departing from its scope. Therefore, the present method and/or system are not limited to the particular implementations disclosed. Instead, the present method and/or system will include all implementations falling within the scope of the appended claims, both literally and under the doctrine of equivalents.

What is claimed is:

1. An injection needle blowoff apparatus, comprising:
a loading surface having a first side configured to contact an injector and having a second side opposite the first side; and
an adjustable blowoff nozzle adjacent the second side of the loading surface, the adjustable blowoff nozzle comprising:
a gas inlet configured to be coupled to a gas supply; and
a gas outlet configured to direct gas from the gas inlet towards a location of a needle of the injector, the gas outlet being adjustable to blow on the needle within a range of distances from the second side of the loading surface.

2. The injection needle blowoff apparatus as defined in claim 1, wherein the adjustable blowoff nozzle is configured to have an adjustable distance from the aperture along a plane of the second side of the loading surface.

3. The injection needle blowoff apparatus as defined in claim 2, wherein the gas outlet is configured to blow at an angle away from the second side of the loading surface, and the gas outlet is configured to blow on the needle at the location based on the distance between the adjustable blowoff nozzle and the needle in in a plane perpendicular to the needle.

4. The injection needle blowoff apparatus as defined in claim 1, wherein the adjustable blowoff nozzle is configured to have an adjustable distance from a tip of the needle in a direction parallel to the needle.

5. The injection needle blowoff apparatus as defined in claim 1, wherein the loading surface comprises a positioning plate having an aperture, extending from a first side of the positioning plate to a second side of the positioning plate.

6. The injection needle blowoff apparatus as defined in claim 5, wherein the adjustable blowoff nozzle is positioned on a first side of the aperture, and further comprising a second adjustable blowoff nozzle positioned on a second side of the aperture.

7. The injection needle blowoff apparatus as defined in claim 6, wherein the second adjustable blowoff nozzle comprises:
a second gas inlet configured to be coupled to the gas supply; and
a second gas outlet configured to direct the gas towards the location of the needle, the second gas outlet being adjustable to blow on the needle within the range of distances.

8. The injection needle blowoff apparatus as defined in claim 5, wherein the aperture has at least one dimension smaller than a corresponding dimension of a body of the injector.

9. The injection needle blowoff apparatus as defined in claim 1, wherein the adjustable blowoff nozzle is configured to have an adjustable angle of the gas outlet.

10. The injection needle blowoff apparatus as defined in claim 1, wherein the adjustable blowoff nozzle comprises a body defining a channel between the gas inlet and the gas outlet, the channel configured to increase at least one of a flow speed of the gas or a pressure of the gas between the gas inlet and the gas outlet.

11. The injection needle blowoff apparatus as defined in claim 1, further comprising control circuitry configured to automatically control a blowoff actuator to adjust a location of the blowoff nozzle.

12. An injector testing device, comprising:
a gas supply;
an injection needle blowoff device, comprising:
a loading surface having a first side configured to contact an injector and having a second side opposite the first side;
an adjustable blowoff nozzle adjacent to the second side of the loading surface, the adjustable blowoff nozzle comprising:
a gas inlet configured to be coupled to the gas supply; and
a gas outlet configured to direct gas from the gas inlet towards a location of a needle of the injector, the gas outlet being adjustable to blow on the needle within a range of distances from the second side of the loading surface; and
control circuitry configured to control the gas supply to output the gas to the adjustable blowoff nozzle.

13. The injector testing device as defined in claim 12, further comprising an injector positioner configured to position the injector, wherein the control circuitry is configured to control the injector positioner to position the injector.

14. The injector testing device as defined in claim 13, wherein the injector positioner is configured to move a body of the injector into contact with the loading surface.

15. The injector testing device as defined in claim 12, further comprising an injector actuator configured to actuate the injector to expel contents of the injector via the needle while the needle of the injector is adjacent the adjustable blowoff nozzle, wherein the control circuitry is configured to control the injector actuator to actuate the injector.

16. The injector testing device as defined in claim 15, wherein the injector actuator is configured to actuate the injector when the body of the injector is in contact with the loading surface.

17. The injector testing device as defined in claim 12, further comprising a collection container configured to collect the contents expelled from the injector.

18. The injector testing device as defined in claim 11, wherein the loading surface comprises a positioning plate having an aperture, extending from a first side of the positioning plate to a second side of the positioning plate, and wherein the adjustable blowoff nozzle is configured to have an adjustable distance from the aperture along a plane of the second side of the positioning plate, the gas outlet is configured to blow at an angle away from the second side of the positioning plate, and the gas outlet is configured to blow on the needle at the location based on the distance between the adjustable blowoff nozzle and the distance from the aperture in a direction parallel to the second side of the positioning plate.

19. The injector testing device as defined in claim 11, further comprising a second adjustable blowoff nozzle configured to direct gas towards the needle.

20. The injector testing device as defined in claim 11, wherein the gas supply comprises a compressed gas source, a pneumatic pump, or a blower.
